(19) United States
(12) Patent Application Publication
(10) Pub. No.: US 2025/0025033 A1
(43) Pub. Date: Jan. 23, 2025

(54) IMAGE STREAMING FOR OPTICAL VIEWING DEVICES

(71) Applicant: Welch Allyn, Inc., Skaneateles Falls, NY (US)

(72) Inventors: David G. Perkins, Tully, NY (US); Danielle R. Endres, Liverpool, NY (US); Lei Guo, Manlius, NY (US); Joshua Hess, Moravia, NY (US); David Villarreal, Fayetteville, NY (US)

(21) Appl. No.: 18/758,134

(22) Filed: Jun. 28, 2024

Related U.S. Application Data
(60) Provisional application No. 63/514,250, filed on Jul. 18, 2023.

(51) Int. Cl. A61B 1/227; A61B 1/05; A61B 5/02; G16H 10/60

(52) U.S. Cl. CPC A61B 1/227; A61B 1/05; A61B 5/02; G16H 10/60

(57) ABSTRACT

An optical viewing system includes an optical viewing device having an eyepiece. The system further includes an imaging device that includes a housing having a bracket that is configured to removably attach the imaging device to the optical viewing device. The imaging device includes a camera configured to capture images viewed through the eyepiece and a display screen configured to display the images captured by the camera. The system further includes a secondary display screen physically separate from the optical viewing device and the imaging device. The secondary display screen pairs with the imaging device to display the images viewed through the eyepiece of the optical viewing device. The pairing between the imaging device and the secondary display screen expires when a condition is satisfied.

### BACKGROUND

Optical viewing devices are used for examining patients as part of routine examinations. Examples of optical viewing devices can include, without limitation, an otoscope for assessing the ears of a patient, an ophthalmoscope for assessing the eyes of a patient, and a dermatoscope for assessing the skin of a patient.

When a physician is using an optical viewing device to view an anatomy of a patient, in some instances, it may also be desirable for the patient and/or a relative of the patient to view images of the anatomy. Also, it may be desirable to share images of an anatomy viewed from an optical viewing device for teaching students, assistants, and apprentices of the physician.

### SUMMARY

In general terms, the present disclosure relates to imaging for optical viewing devices. In one possible configuration, an imaging device includes a display screen for displaying images viewed through an eyepiece of an optical viewing device, and the imaging device streams the images to a secondary display screen. Various aspects are described in this disclosure, which include, but are not limited to, the following aspects.

One aspect relates to an optical viewing system, comprising: an optical viewing device having an eyepiece; an imaging device including: a housing having a bracket that is configured to removably attach the imaging device to the optical viewing device; a camera configured to capture images viewed through the eyepiece; and a display screen configured to display the images captured by the camera; and a secondary display screen physically separate from the optical viewing device and the imaging device, the secondary display screen pairing with the imaging device to display the images viewed through the eyepiece of the optical viewing device, and the pairing between the imaging device and the secondary display screen expires when a condition is satisfied.

Another aspect relates to an imaging device for use with an optical viewing device, the imaging device comprising: a housing having a bracket for removably attaching the imaging device to the optical viewing device; a camera for capturing images viewed from an eyepiece of the optical viewing device; a display screen for displaying the images captured by the camera; and a communications device for streaming the images viewed from the eyepiece of the optical viewing device to a secondary display screen separately located from the optical viewing device and the imaging device, wherein a pairing between the imaging device and the secondary display screen expires when a condition is satisfied.

Another aspect relates to an optical viewing system, comprising: an optical viewing device having an eyepiece; and an imaging device including: a housing having a bracket for removably attaching the imaging device to the optical viewing device; a camera for capturing images viewed through the eyepiece; a display screen for displaying the images captured by the camera; and a communications device for streaming the images viewed from the eyepiece of the optical viewing device to a secondary display screen separately located from the optical viewing device and the imaging device, wherein a pairing between the imaging device and the secondary display screen expires when a condition is satisfied.

A variety of additional aspects will be set forth in the description that follows. The aspects can relate to individual features and to combinations of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of an optical viewing system being used by a clinician to perform a physical assessment of a patient.

FIG. 2 illustrates another example of the optical viewing system of FIG. 1.

FIG. 3 illustrates an example of an imaging device displaying an image captured from an eyepiece of an optical viewing device of the optical viewing system of FIG. 1.

FIG. 4 illustrates another example of the optical viewing system of FIG. 1.

FIG. 5 schematically illustrates an example of a method of assisting a physical assessment of the patient that can be performed by the optical viewing system of FIG. 1.

FIG. 6 shows examples of different types of optical viewing devices that can be used with the optical viewing system of FIG. 1.

FIG. 7 is an isometric view of an example of an optical viewing device of the optical viewing system of FIG. 1, the optical viewing device shown from a clinician perspective.

FIG. 8 is another isometric view of the optical viewing device of FIG. 7, the optical viewing device shown from a patient perspective.

FIG. 9 is an isometric view of an example of the imaging device of the optical viewing system of FIG. 1, the imaging device is shown attached to an ophthalmoscope, and the imaging device is shown from the clinician perspective.

FIG. 10 is a front isometric view of the imaging device of FIG. 9.

FIG. 11A is a front view of the imaging device of FIG. 9.

FIG. 11B is a rear view of the imaging device of FIG. 9.

FIG. 11C is a top view of the imaging device of FIG. 9.

FIG. 12 is an isometric view showing a camera of the imaging device of FIG. 9.

FIG. 13 is a front isometric view of an example of the imaging device of FIG. 9 attached to an optical viewing device.

FIG. 14 is a rear isometric view of the imaging device of FIG. 9 attached to the optical viewing device.

FIG. 15 is an isometric view of another example of the imaging device of the optical viewing system of FIG. 1, the imaging device is shown attached to an ophthalmoscope, and the imaging device is shown from the clinician perspective.

FIG. 16 is an isometric view of the imaging device of FIG. 15 before attachment to the ophthalmoscope, the imaging device is shown from the patient perspective.

FIG. 17 illustrates an exemplary architecture of a computing device of the imaging device shown in any of the above figures.

### DETAILED DESCRIPTION

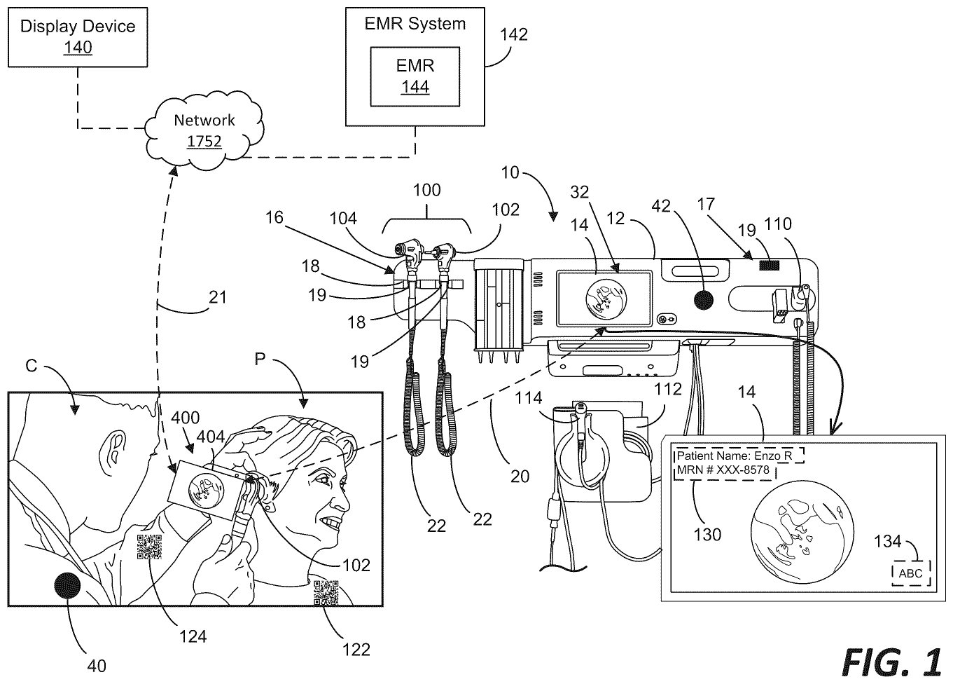

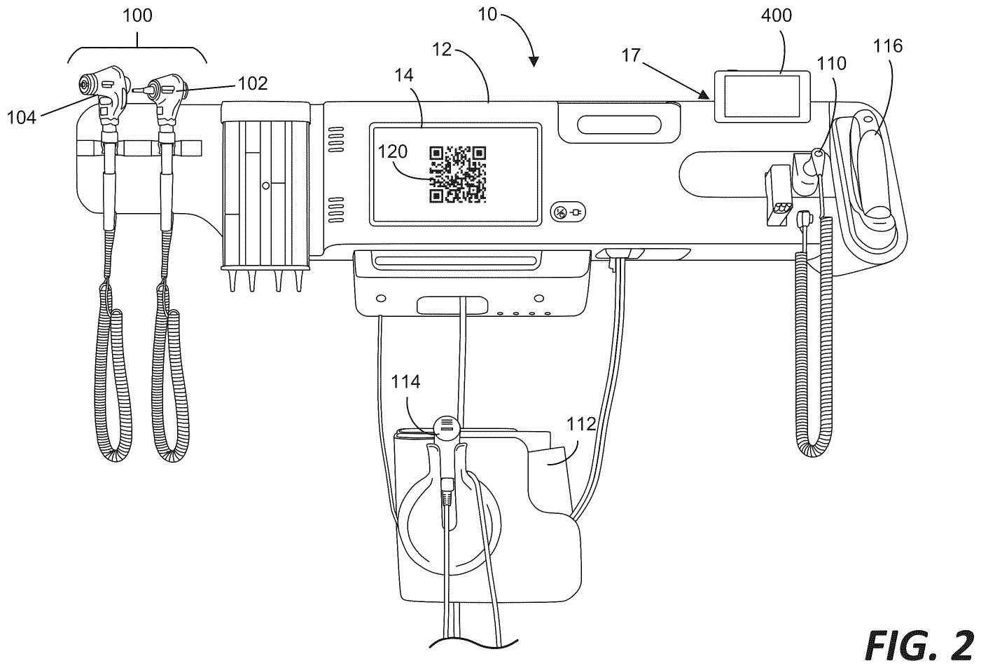

FIG. 1 illustrates an example of an optical viewing system 10 being used by a clinician C to perform a physical assessment of a patient P. FIG. 2 illustrates another example of the optical viewing system 10 before being used by the clinician C to perform the physical assessment. As shown in FIGS. 1 and 2, the optical viewing system 10 includes one or more optical viewing devices 100, and an imaging device 400 configured for use with the one or more optical viewing devices 100. The imaging device 400 can removably attach to each of the one or more optical viewing devices 100 to capture images from an eyepiece of the optical viewing devices, and to display the images on a display screen 404 for viewing by the clinician C.

The optical viewing system 10 includes a secondary display screen 14 that is physically separate from the optical viewing devices 100 and the imaging device 400. The secondary display screen 14 is communicatively coupled to the optical viewing devices 100 and the imaging device 400 such as through one or more wireless connections. As will be described in more detail, the secondary display screen 14 displays the images captured by the imaging device 400 from the eyepiece of the optical viewing device 100 for viewing by the patient P, or by someone else present in the room where the optical viewing system 10 is positioned such as a relative or guardian of the patient P. In further examples, a student, an assistant, or an apprentice of the clinician C can view images displayed on the secondary display screen 14 that are captured by the imaging device 400 from the eyepiece of the optical viewing device 100.

The clinician C can view the images captured from the optical viewing device 100 on the display screen 404 of the imaging device 400, and the patient P and other persons present in the room can simultaneously view the images on the secondary display screen 14. This allows the clinician C to explain a diagnosis or a prescribed treatment while the patient P and the other persons in the room view the images on the secondary display screen 14. This can improve patient engagement and satisfaction because the patient P can view their anatomy while receiving from the clinician C a diagnosis or a prescribed treatment.

As shown in FIG. 1, the secondary display screen 14 receives the images from the imaging device 400 through a wireless connection 20. Examples of the wireless connection 20 can include Wi-Fi, and other suitable wireless connections for transferring image and video data.

As further shown in FIG. 1, the imaging device 400 can connect to a network 1752 via a wireless connection 21. In some examples, the imaging device 400 transmits via the network 1752 the images to the secondary display screen 14.

In some examples, the imaging device 400 transmits via the network 1752 the images to a display device 140 that is remotely located from the optical viewing devices 100 and the secondary display screen 14. In such examples, a remote clinician can review the images on the display device 140 to perform overread services, telemedicine, and the like.

In further examples, the imaging device 400 transmits via the network 1752 the images to an electronic medical record (EMR) system 142 such as to store the images in an electronic medical record (EMR) 144 of the patient P. The images when stored in the EMR 144 of the patient are accessible by the clinician C or by another clinician at any time such as during a follow-up visit to the office of the clinician C, or when the patient P is referred to another clinician for a further opinion or follow-up. Also, the patient P can access the images when stored in the EMR 144 of the patient such as through a patient portal.

In some examples, the secondary display screen 14 is a touchscreen such that it can both display the images received from the imaging device 400, and receive inputs from a user such as the clinician C. The secondary display screen 14 can include a true color multi-touch screen (in-plane switching (IPS), or light-emitting diode (LED)). The secondary display screen 14 can also include features such as screen automatic turn-off and wake-up such as by pressing a power button, by tapping the secondary display screen 14, by detecting a pairing between the secondary display screen 14 and the imaging device 400, or by detecting some other type of action associated with the one or more optical viewing device 100 and the imaging device 400.

In the example shown in FIGS. 1 and 2, the secondary display screen 14 is included on a wall mounted unit 12 that includes additional devices for physically assessing the patient P. In alternative examples, the secondary display screen 14 can be included on a different type of unit or device such that FIGS. 1 and 2 are provided by way of illustrative example.

The wall mounted unit 12 includes a storage area 16 for the one or more optical viewing devices 100. The storage area 16 includes holders 18 for holding the optical viewing devices 100 on the wall mounted unit 12. The storage area 16 can include more than one holder for holding more than one type of optical viewing device. In the example shown in FIGS. 1 and 2, the storage area 16 includes a holder for a first type of optical viewing device such as an otoscope 102, and another holder for a second type of optical viewing device such as an ophthalmoscope 104. In further examples, the storage area 16 on the wall mounted unit 12 holds additional types and/or quantities of optical viewing devices including a dermatoscope.

Additionally, in some examples, the wall mounted unit 12 can include storage area 17 for holding the imaging device 400 when not being used. In some further examples, the wall mounted unit 12 recharges the imaging device 400 when held in the storage area 17.

In some examples, such as the one shown in FIGS. 1 and 2, the one or more optical viewing devices 100 are tethered to the wall mounted unit 12 such as by cables 22. In such examples, a cable 22 supplies electrical power to an optical viewing device 100 such as to power the components of the device. The optical viewing devices 100 can each include a rechargeable battery, such as a rechargeable battery stored in a handle of the optical viewing device. The rechargeable battery of each optical viewing device 100 is charged when connected to a cable 22 of the wall mounted unit 12, or when held inside a well of a desk charging unit (not shown).

As further shown in FIGS. 1 and 2, the wall mounted unit 12 further includes one or more devices for obtaining physiological parameter measurements of the patient P. For example, the wall mounted unit 12 can include a first type of thermometer 110 (e.g., mouth probe thermometer), a blood pressure monitor 112, a pulse oximeter 114, and a second type of thermometer 116 (e.g., car probe thermometer, see FIG. 2). Additional types of devices for measuring physiological parameters, or a fewer number of devices, may be included on the wall mounted unit 12 for obtaining physiological parameter measurements of the patient.

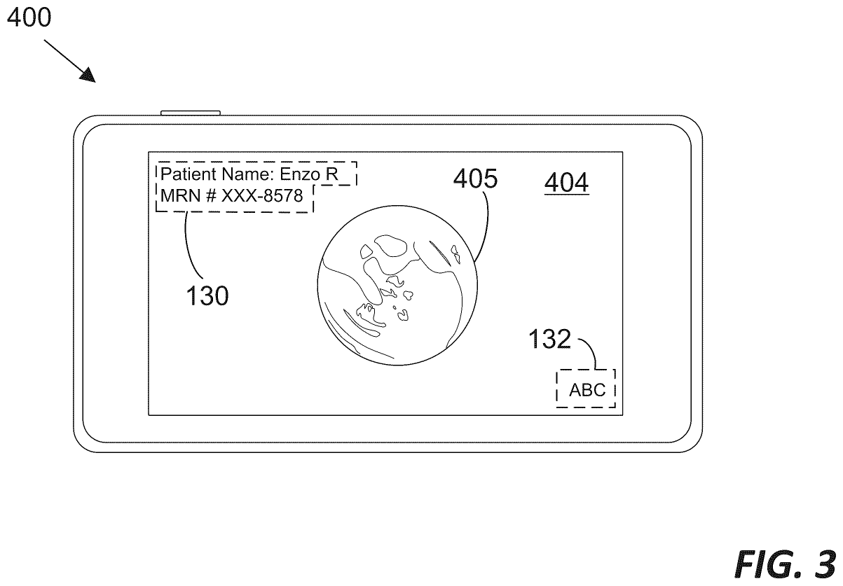

FIG. 3 illustrates an example of an image 405 captured from the eyepiece of the optical viewing device 100 displayed on the imaging device 400. As will be described in more detail further below, the imaging device 400 includes a camera 410 that captures the image 405 from the eyepiece of the optical viewing device 100. Also, the camera 410 can be used to scan one or more machine-readable labels, as well be described in more detail further below.

In the illustrative example shown in FIG. 3, the image 405 displayed on the display screen 404 of the imaging device 400 includes an car canal anatomy. Thus, in this example, the imaging device 400 is attached to the otoscope 102. When the imaging device 400 is attached to the ophthalmoscope 104, the image 405 displayed on the display screen 404 of the imaging device 400 is of the eye, and the eye image can be streamed to the secondary display screen 14. When the imaging device 400 is attached to a dermatoscope, the image 405 displayed on the display screen 404 of the imaging device 400 is of the skin, and the skin image can be streamed to the secondary display screen 14. Additional types of images can be displayed on the display screen 404 of the imaging device 400 based on the type of optical viewing device that the imaging device 400 is attached to, and such images can be similarly streamed to the secondary display screen 14 as desired by the clinician C during a physical assessment exam.

The imaging device 400 is paired to the secondary display screen 14 before the imaging device 400 can stream the image 405 captured from the eyepiece of the otoscope 102 to the secondary display screen 14. The pairing between the imaging device 400 and the secondary display screen 14 maintains the privacy of protected health information (PHI) of the patient P because the pairing prevents the image 405 from being displayed, whether unintentionally or not, on another display device that can be viewed by someone who is not the patient P or a relative.

In the illustrative example shown in FIG. 2, the imaging device 400 can be paired to the secondary display screen 14 by scanning a machine-readable label 120 displayed on the secondary display screen 14. In some examples, the machine-readable label 120 is displayed on the secondary display screen 14 when the clinician C selects a control on the imaging device 400 for pairing the imaging device 400 to the secondary display screen 14.

In further examples, the machine-readable label 120 is automatically displayed on the secondary display screen 14 when the imaging device 400 is removed from the storage area 17. In further examples, the machine-readable label 120 is automatically displayed on the secondary display screen 14 when an optical viewing device 100 is removed from the storage area 16. In another example, the machine-readable label 120 is automatically displayed on the secondary display screen 14 when both an optical viewing device 100 and the imaging device 400 are removed from their respective storage areas 16, 17. Additional examples of triggering automatic display of the machine-readable label 120 on the secondary display screen 14 are possible.

The wall mounted unit 12 can include sensors 19 to detect when the optical viewing device 100 and the imaging device 400 are removed from their respective storage areas. The sensors 19 can similarly detect when these devices are returned to their respective storage areas. The sensors 19 can be positioned inside or proximate to the storage areas 16, 17 on the wall mounted unit 12. Examples of the sensors 19 can include proximity sensors, reed switches, Hall effect sensors, magnetometers, pressure sensors, and other similar types of sensors for detecting the presence of the optical viewing device 100 and the imaging device 400.

In the example shown in FIG. 2, the machine-readable label 120 displayed on the secondary display screen 14 is a quick response (QR) code. Alternatively, the machine-readable label 120 displayed on the secondary display screen 14 can include a linear or one-dimensional barcode, and other machine-readable forms of visually representing data. In yet further examples, the machine-readable label 120 can include a label that is physically attached to an exterior of the wall mounted unit 12 such as sticker or other type of physical label.

In some examples, the machine-readable label 120 is dynamically updated at predetermined intervals of time. This ensures that only fresh, newly generated machine readable labels are used to pair the imaging device 400 to the secondary display screen 14. This can mitigate opportunities for a person to copy the machine-readable label 120 for pairing the imaging device 400 to a third party device, and can thus further enhance protection of PHI.

In further examples, an ID associated with the imaging device 400 can be displayed on the secondary display screen 14 for selection by the clinician C to pair the secondary display screen 14 to the imaging device 400. Alternatively, an ID associated with the secondary display screen 14 can be displayed on the display screen 404 of the imaging device 400 for selection by the clinician C to pair the imaging device 400 to the secondary display screen 14.

In further illustrative examples, the imaging device 400 can be paired to the secondary display screen 14 by using wireless tags such as radio-frequency identification (RFID) tags, or similar types of wireless tags. In such examples, when the imaging device 400 is positioned in close proximity to the secondary display screen 14 such as when the imaging device 400 is brought into the same room or area where the secondary display screen 14 is located, the imaging device 400 and the secondary display screen 14 can recognize each other, and automatically pair together. In further examples, when the imaging device 400 touches the secondary display screen 14, the imaging device 400 and the secondary display screen 14 can recognize each other, and automatically pair together. Additional examples of pairing the imaging device 400 to the secondary display screen 14 are possible.

In some further examples, the pairing between the imaging device 400 and the secondary display screen 14 expires when a condition is satisfied. For example, the pairing between the imaging device 400 and the secondary display screen 14 can expire when the optical viewing device 100 is returned to the storage area 16 and/or the imaging device 400 is returned to the storage area 17. The sensors 19 can be used to detect when the optical viewing device 100 and the imaging device 400 are returned from their respective storage areas. Alternately, or additionally, the pairing may disconnect when the imaging device 400 is detached from the optical viewing device 100 or when the imaging device 400 is turned off or put in standby mode.

As another example, the pairing between the imaging device 400 and the secondary display screen 14 can expire after a predetermined period of time elapses. For example, pairing between the imaging device 400 and the secondary display screen 14 can expire after a predetermined period of time that is sufficient for the clinician C to perform an assessment of an anatomy using any one of the optical viewing devices 100, and that is short enough such that the clinician C is not likely to use the optical viewing device 100 on another patient. For example, the predetermined period of time can be 10 minutes. Additional periods of time for terminating the pairing between the imaging device 400 and the secondary display screen 14 are possible.

The expiration of the pairing between the imaging device 400 and the secondary display screen 14 further mitigates loss of PHI by requiring reestablishment of the pairing each time the imaging device 400 is used. Thus, a default mode of operation of the imaging device 400 does not include streaming the images 405 to the secondary display screen 14. Instead, the images 405 are streamed from the imaging device 400 to the secondary display screen 14 only when the clinician C takes affirmative action to do so by pairing the imaging device 400 to the secondary display screen 14 such as by scanning the machine-readable label 120.

As shown in FIG. 1, the imaging device 400 can scan a machine-readable label 122 associated with the patient P for determining an identity (ID) 130 of the patient P. In other examples, the ID 130 of the patient P can be determined by detecting digital data from a tag worn by the patient P such as by using RFID, or other wireless technologies.

In some examples, the ID 130 is displayed on the secondary display screen 14 (see FIG. 1). In some further examples, the ID 130 can also be displayed on the display screen 404 of the imaging device 400 (see FIG. 3). The ID 130 can further enhance the protection of PHI because the clinician C and/or the patient P can verify that the images displayed on the secondary display screen 14 belong to the patient P, and not to another patient.

In some examples, the information included in the ID 130 is truncated or at least partially concealed such that enough information is provided to correctly identify the patient P, but without providing full details. For example, certain letters of the patient's name can be obscured (e.g., L**** RIC****). As a further example, the patient P's first name and first initial of their last name can included in the ID 130. Additionally, or alternatively, only the last several digits of the patient P's medical record number (MRN) can be displayed in the ID 130.

In some examples, the display screen 404 of the imaging device 400 displays a first code 132 (FIG. 3), and the secondary display screen 14 displays a second code 134 (FIG. 1). Illustrative examples of the first and second codes 132, 134 can include alphanumeric sequences, clinic or room names, icons or images, and the like. In such examples, the first and second codes 132, 134 provide confirmation that the imaging device 400 is correctly paired to the secondary display screen 14. For example, the pairing between the imaging device 400 and the secondary display screen 14 is confirmed when the second code 134 matches the first code 132. The first and second codes 132, 134 improve the protection of PHI because they provide confirmation that the images displayed on the secondary display screen 14 are from the imaging device 400.

The imaging device 400 can further scan a machine-readable label 124 associated with the clinician C. In some examples, the clinician C is authenticated by scanning the machine-readable label 124 to identify the clinician C, and thereafter the optical viewing system 10 checks whether the clinician C is authorized to use the secondary display screen 14, the one or more optical viewing devices 100, and/or the imaging device 400. In some examples, the imaging device 400 is blocked from streaming the images to another device unless the clinician Cis authorized to use the secondary display screen 14, the one or more optical viewing devices 100, and/or the imaging device 400. In some further examples, the imaging device 400 is not operable unless the clinician C is authorized to use the secondary display screen 14, the one or more optical viewing devices 100, and/or the imaging device 400.

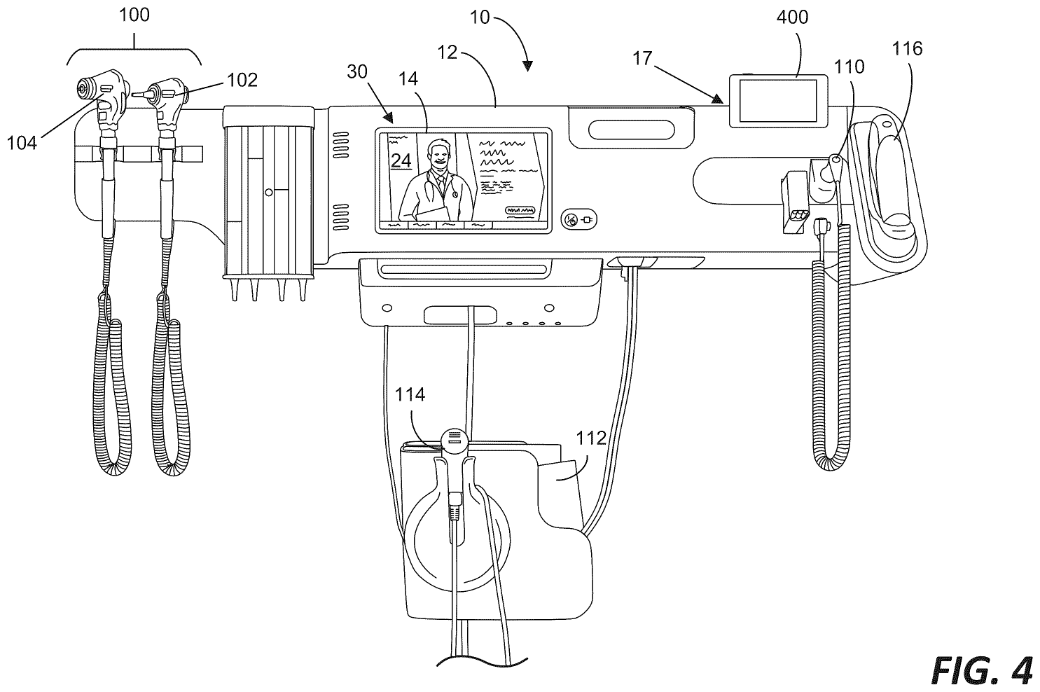

FIG. 4 illustrates another example of the optical viewing system 10 when not being used by the clinician C to perform a physical assessment. For example, the optical viewing system 10 operates in a patient mode 30 when not being used by the clinician C. As shown in FIG. 4, the secondary display screen 14 displays content 24 that is unrelated to the optical viewing devices 100 and the imaging device 400 when operating in the patient mode 30. For example, the content 24 displayed on the secondary display screen 14 while operating in the patient mode 30 can include patient appropriate content such as educational materials, entertainment for kids, and/or advertisements for the practice of the clinician C or other entities.

In contrast, when the optical viewing system 10 is being used by the clinician C to perform a physical assessment (e.g., see FIG. 1), the optical viewing system 10 operates in a clinician mode 32. When in the clinician mode 32, the secondary display screen 14 can display content that is relevant to the physical assessment of the patient P such as the images 405 captured by the imaging device 400 from an eye piece of an optical viewing device 100. In further examples, when in the clinician mode 32, the secondary display screen 14 can display physiological parameter measurements obtained by the other devices included on the wall mounted unit 12 such as temperature, blood pressure, blood oxygen saturation (SpO2), pulse, electrocardiogram (ECG or EKG), and other physiological parameter measurements.

The optical viewing system 10 can automatically switch from the patient mode 30 to the clinician mode 32 when an event is detected. For example, the optical viewing system 10 can automatically switch from the patient mode 30 to the clinician mode 32 when removal of a device from its designated storage area is detected on the wall mounted unit 12 such as removal of an optical viewing device from the storage area 16, removal of the imaging device 400 from the storage area 17, or removal of the first type of thermometer 110, the blood pressure monitor 112, the pulse oximeter 114, the second type of thermometer 116, and other physiological parameter measuring devices from their respective storage areas on the wall mounted unit.

In further examples, the optical viewing system 10 can automatically switch from the patient mode 30 to the clinician mode 32 when the clinician C is detected to be in close proximity to the optical viewing system 10. For example, the clinician C can wear a tag 40 (see FIG. 1) that is detectable by a reader device 42 of the optical viewing system 10 when the clinician C is in close proximity to the optical viewing system 10. When triggered by an electromagnetic interrogation pulse from the reader device 42, the tag 40 worn by the clinician C transmits digital data that identifies the clinician C back to the reader device. In some examples, the tag 40 worn by the clinician C is a RFID tag, and the reader device 42 is a RFID reader.

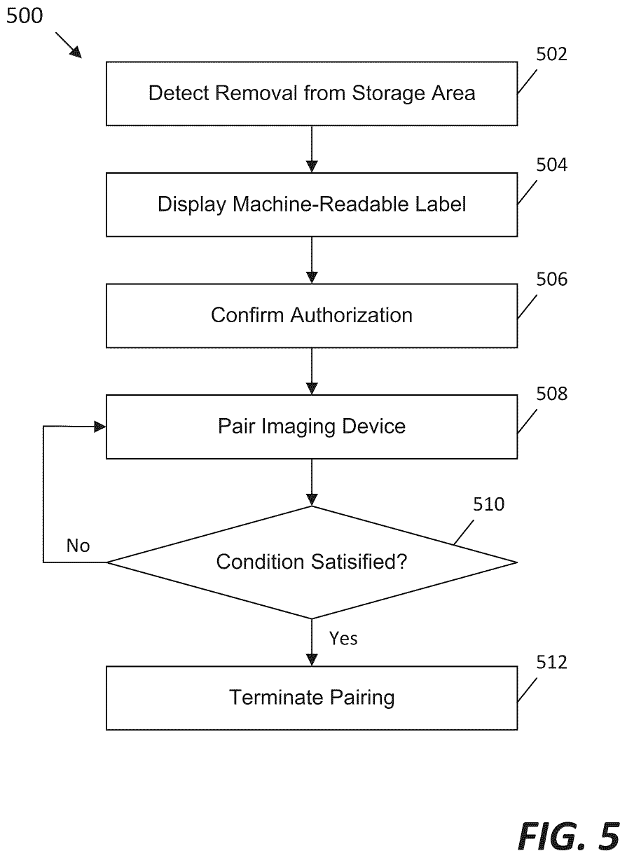

FIG. 5 schematically illustrates an example of a method 500 of assisting a physical assessment of the patient P. The method 500 can be performed by the optical viewing system 10. As shown in FIG. 5, the method 500 includes an operation 502 of detecting removal of the imaging device 400 from the storage area 17. As described above, the storage area 17 includes the sensor 19 to detect when the imaging device 400 is removed from the storage area 17.

Next, the method 500 includes an operation 504 of displaying the machine-readable label 120 on the secondary display screen 14. This operation is shown in FIG. 2. In some examples, operation 504 includes displaying the machine-readable label 120 in response to detecting removal of the imaging device 400 from the storage area 17.

In alternative examples, operation 504 includes displaying the machine-readable label 120 in response to receiving a request from the imaging device 400 to pair with the secondary display screen 14. For example, the request can be generated when the clinician C selects a control on imaging device 400 to pair the imaging device 400 to the secondary display screen 14.

In further examples, operation 504 includes displaying the machine-readable label 120 in response to receiving a request from the secondary display screen 14 to pair with the imaging device 400. For example, the request can be generated when the clinician C selects a control on the secondary display screen 14 or elsewhere on the wall mounted unit 12 to pair the secondary display screen 14 to the imaging device 400.

In some examples, operation 504 includes autodetecting available secondary screens and identifying the available secondary screens on the display screen 404 of the imaging device 400. When a user selects an available secondary screen on the display screen 404, the method 500 can include displaying the machine-readable label 120 on the secondary display screen 14.

Next, the method 500 can include an operation 506 of confirming whether the secondary display screen 14 is allowed to be paired with the imaging device 400. Operation 506 can include scanning the machine-readable label 120 on the secondary display screen 14 using the camera 410 of the imaging device, and comparing machine-readable data acquired from the label to digital data identifying the clinician C which can be acquired from the camera 410 of the imaging device 400 scanning the machine-readable label 124 associated with the clinician C, or from the reader device 42 detecting digital data transmitted by the tag 40 worn by the clinician C.

Once the clinician C is identified by the optical viewing system 10, operation 506 can include checking whether the clinician C is authorized to use the imaging device 400 and/or the secondary display screen 14. When the clinician C is not authorized to use the imaging device 400 and/or the secondary display screen 14, the imaging device 400 is blocked from streaming images to the secondary display screen 14. Otherwise, when the clinician C is authorized to use the imaging device 400 and/or the secondary display screen 14, the method 500 can proceed to an operation 508 of pairing the secondary display screen 14 to the imaging device 400.

As shown in FIG. 1, when the secondary display screen 14 is paired to the imaging device 400, the images captured by the camera of the imaging device 400 are displayed on the secondary display screen 14. In some examples, operation 518 includes casting a video feed captured by the camera of the imaging device 400 onto the secondary display screen 14. As used herein, the term “casting” means to distribute a stream of data, images, and sound. The casting performed in operation 508 can utilize open protocols such as Chromecast® and others. The casting causes the stream of images and data displayed on the display screen 404 of the imaging device 400 to be mirrored on the secondary display screen 14.

Operation 508 enables the patient P or someone else in the room with the patient P and the clinician C can view the images on the secondary display screen 14, while the clinician C is viewing the same images on the imaging device 400. In some examples, the images are displayed on the secondary display screen 14 simultaneously while the physical assessment is being performed by the clinician C using the optical viewing device. Alternatively, the images can be displayed on the secondary display screen 14 after the physical assessment is completed by the clinician C such as when it is desirable for the clinician C to discuss the results from the physical assessment including a diagnosis, and a recommendation for treatment.

The method 500 includes an operation 510 of determining whether a condition is satisfied for terminating the pairing between the secondary display screen 14 and the imaging device 400. As an example, operation 510 can include detecting that the imaging device 400 is returned to the storage area 17, which suggests that the physical assessment of the patient P is completed. The sensor 19 can be used to detect when the imaging device 400 is returned to the storage area 17. As another example, operation 510 can include determining that the imaging device 400 is idle for longer than a predetermined period of time such as when the imaging device 400 goes into a sleep mode, which suggests that the physical assessment of the patient P is completed. As a further example, the display screen 404 can display an icon that is selectable (in examples where the display screen 404 is a touchscreen), to quickly disable the casting. In further examples, the secondary display screen 14 can display an icon that is selectable (in examples where the secondary display screen 14 is a touchscreen), to quickly disable the casting.

When the operation 510 determines that the condition is not satisfied (i.e., “No” in operation 510), the method 500 can return to operation 508 to maintain the pairing between the secondary display screen 14 and the imaging device 400, which allows the secondary display screen 14 to continue to receive the images captured by the imaging device 400. Otherwise, when the operation 510 determines that the condition is not satisfied (i.e., “No” in operation 510), the method 500 can proceed to an operation 512 of terminating the pairing between the secondary display screen 14 and the imaging device 400. As discussed above, when the imaging device 400 is no longer paired with the secondary display screen 14, the secondary display screen 14 is not able to display the images captured by the imaging device 400.

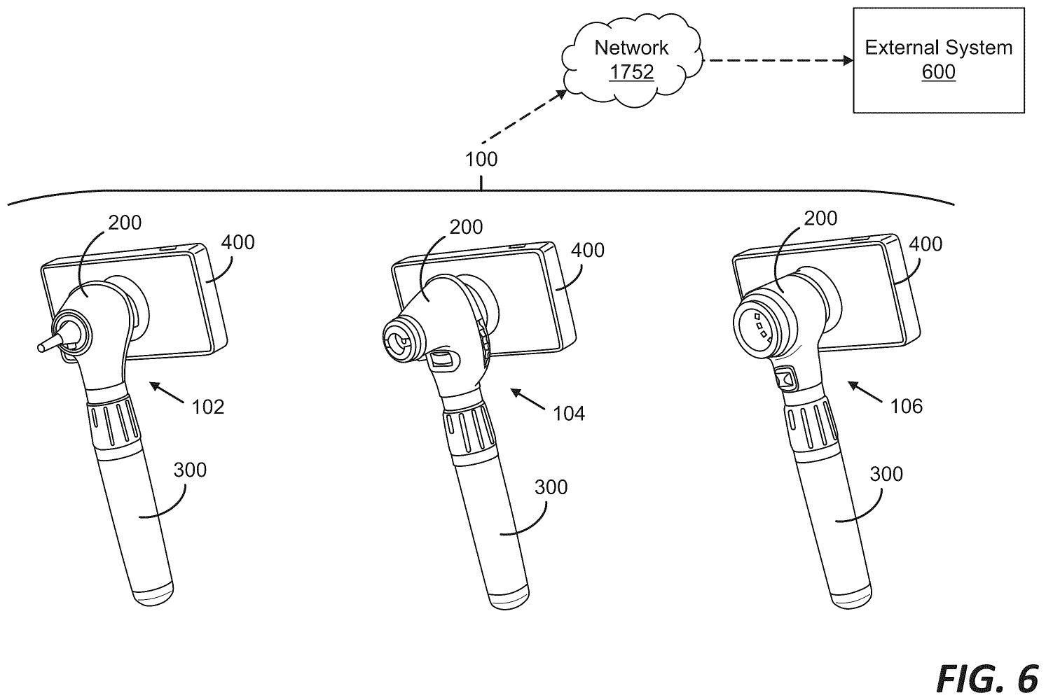

FIG. 6 shows examples of different types of optical viewing devices 100 that can be used with the optical viewing system 10. For example, the optical viewing devices 100 include a first type of optical viewing device such as the otoscope 102, a second type of optical viewing device such as the ophthalmoscope 104, and a third type of optical viewing device such as a dermatoscope 106. Additional types of the optical viewing devices 100 are possible, and the disclosure provided herein is not limited to otoscopes, ophthalmoscopes, and dermatoscopes.

As shown in FIG. 6, each type of optical viewing device 100 includes an instrument head 200 attached to an instrument handle 300. The instrument head 200 can include a light source and optics for viewing an anatomical area of interest through an eyepiece. The instrument handle 300 can include a power source that powers the light source and other components of the instrument head 200. For example, the instrument handle 300 can include rechargeable batteries.

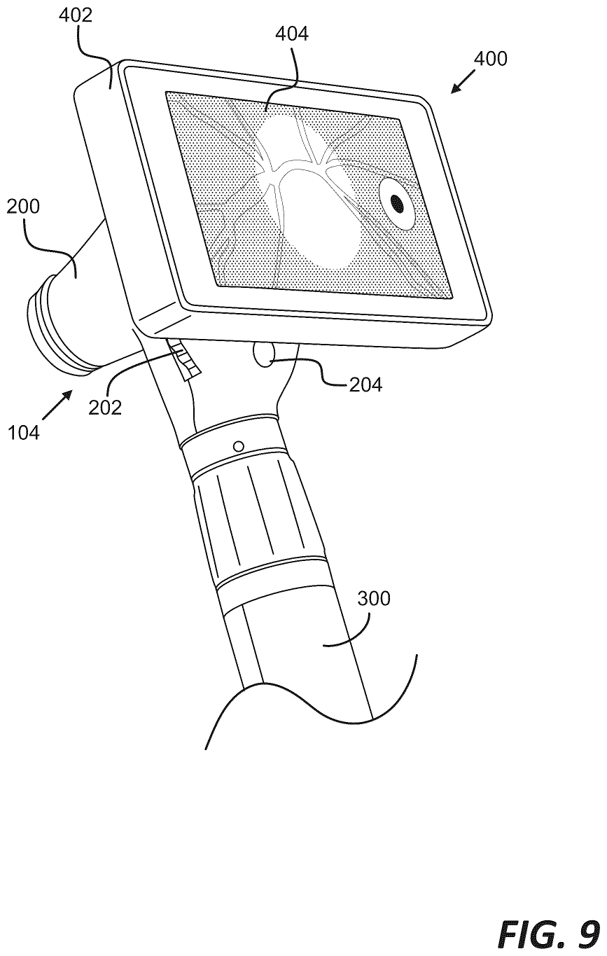

As further shown in FIG. 6, the imaging device 400 can attach to the instrument head 200 of each type of optical viewing device 100. The imaging device 400 is a portable, battery powered camera that can record high quality image frames and videos from the optical viewing devices 100, providing digital imaging solutions. For example, the imaging device 400 captures images through an eyepiece of the instrument head 200 for display on a display screen 404 (see FIG. 9) for viewing by the clinician C. The images captured by the imaging device 400 can be analyzed by algorithms (including artificial intelligence algorithms) for disease screening, and the images can be stored in the EMR 144 of the patient P.

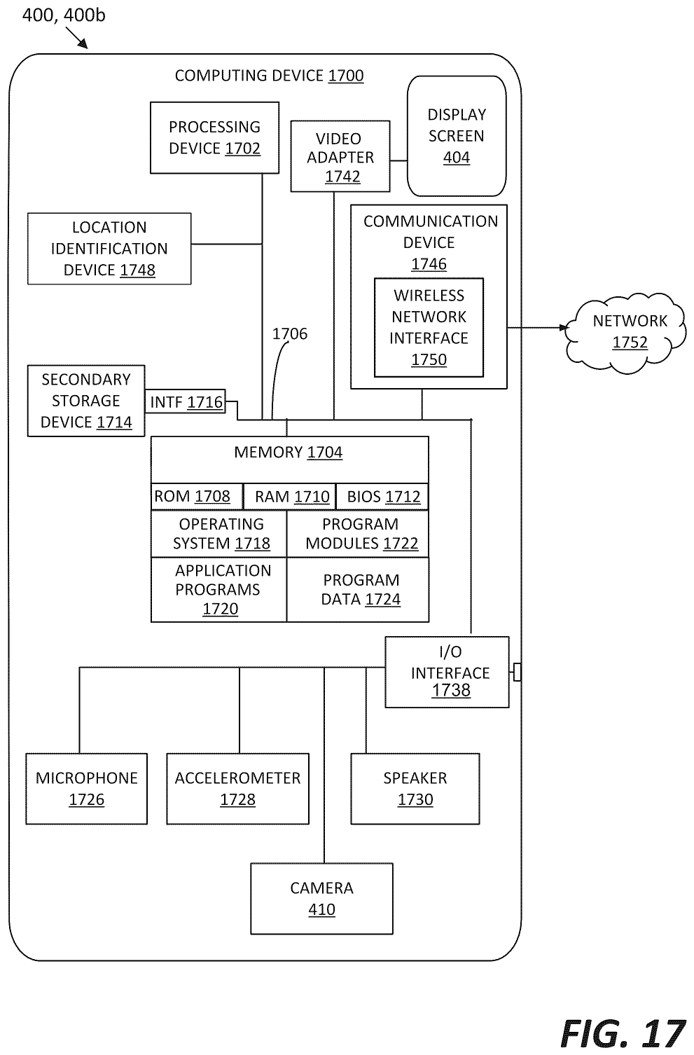

In some examples, the imaging device 400 transmits images, videos, and other data to an external system 600 such as the wall mounted unit 12, or a remote server. The external system 600 analyzes the images, videos, and data received from the imaging device 400 to generate one or more results for transmission back to the imaging device 400. The external system 600 can be remotely located with respect to the optical viewing device 100 and the imaging device 400. In some examples, the external system 600 includes a cloud server. The imaging device 400 can communicate with the external system 600 via a network 1752 (see also FIG. 17).

The algorithms (including artificial intelligence algorithms) for disease screening can be executed on either or both of the imaging device 400 and the external system 600. In some examples, the external system 600 may also host storage of the images, videos, and other data received from the imaging device 400. In further examples, the external system 600 can host the EMR 144 of the patient. In yet further examples, the external system 600 may provide connectivity to other external systems and servers having image storage, or that host the EMR.

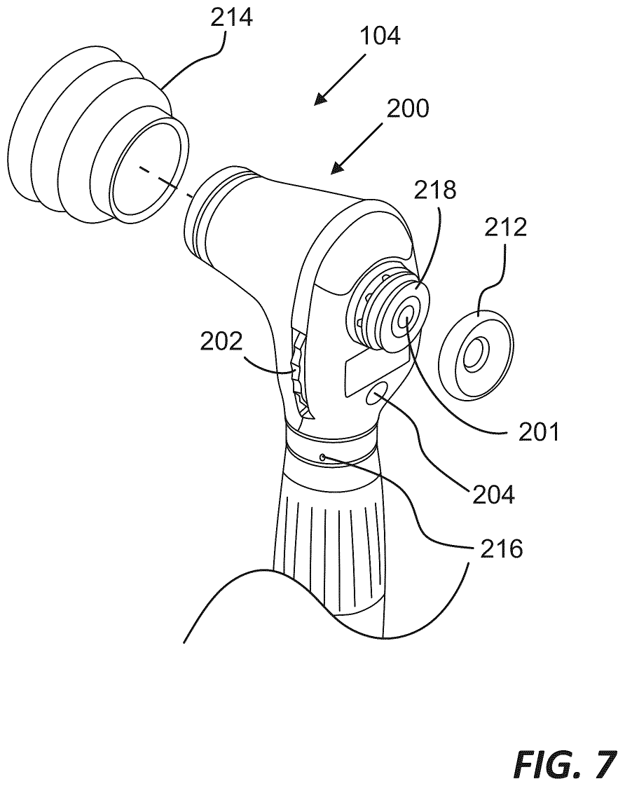

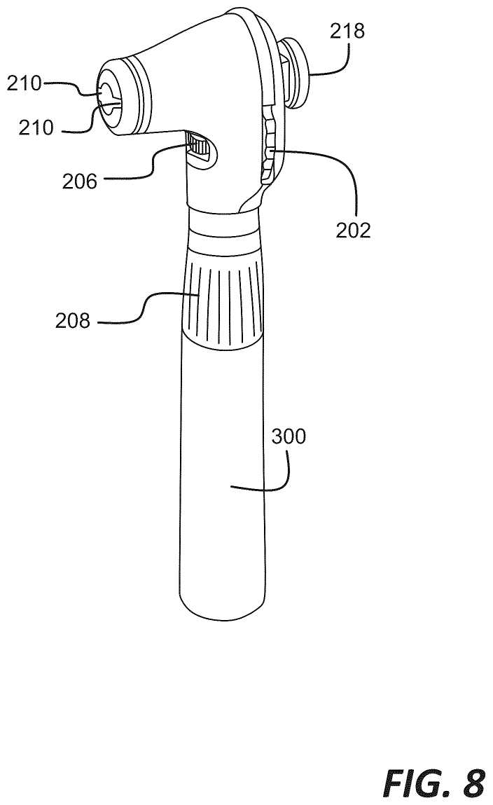

FIGS. 7 and 8 are isometric views of an example of the ophthalmoscope 104. In FIG. 7, the ophthalmoscope 104 is shown from a physician perspective. In FIG. 8, the ophthalmoscope 104 is shown from a patient perspective. While the following description refers to the ophthalmoscope 104 shown in FIGS. 7 and 8, the otoscope 102, the dermatoscope 106, and other types of optical viewing devices can include similar components and features.

Referring now to FIGS. 7 and 8, the ophthalmoscope 104 includes a diopter focus wheel 202 and a diopter readout 204. The diopter focus wheel 202 can be used by the clinician C to adjust a focus of an eyepiece 201, which can be displayed on the diopter readout 204. The eyepiece 201 includes one or more optical components that magnify a view of an anatomical structure. The one or more optical components of the eyepiece 201 can include one or more lenses, filters, and the like that are selectable and/or adjustable by the diopter focus wheel 202 to increase the magnification or decrease the magnification viewed through the eyepiece 201.

In some examples, the diopter focus wheel 202 can be used to correct refractive errors of the clinician C and/or the patient P. For example, the diopter focus wheel 202 can be used to provide a positive dioptric value to accommodate for hyperopia eyesight (farsightedness) of the clinician C and/or the patient P, and to provide a negative dioptric value to accommodate for myopia eyesight (nearsightedness) of the clinician C and/or the patient P. The dioptric values that are adjusted by the diopter focus wheel 202 are displayed in the diopter readout 204. When used with the imaging device 400, the diopter focus wheel 202 can also allow corrections for the patient P's vision. The diopter focus wheel 202 also allows for up-close focusing of the images captured by the camera 410 for display on the display screen 404 of the imaging device 400 and the secondary display screen 14, such as for viewing or imaging the patient P's cornea or sclera.

The ophthalmoscope 104 can include a filter wheel 206 to select a filter for viewing through the eyepiece 201. For example, the filter wheel 206 can be used to select a reticle target to help measure the optic disc, a cobalt blue filter to help detect corneal abrasions, a red-free filter to highlight blood vessels and other sources of blood, and additional types of filters.

The ophthalmoscope 104 can further include a light control 208 for controlling illumination from the light source, disc alignment lights 210 (e.g., red for right eye exams; yellow for left eye exams), an eyepiece bumper 212, an optional patient eye cup 214, an optional locking collar 216, and an eyepiece housing 218. As will be described in more detail below, the imaging device 400 includes a bracket that removably attaches to the eyepiece housing 218 for securing the imaging device 400 to the instrument head 200.

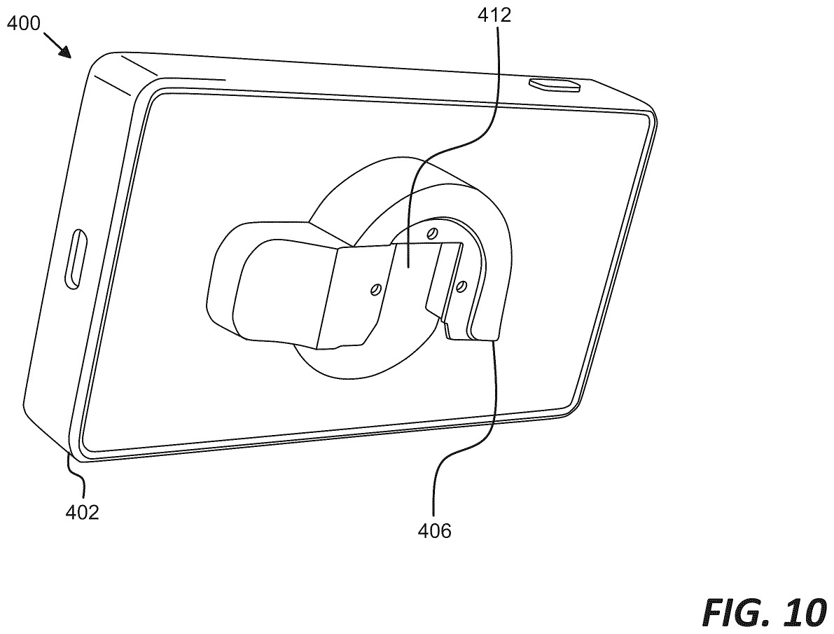

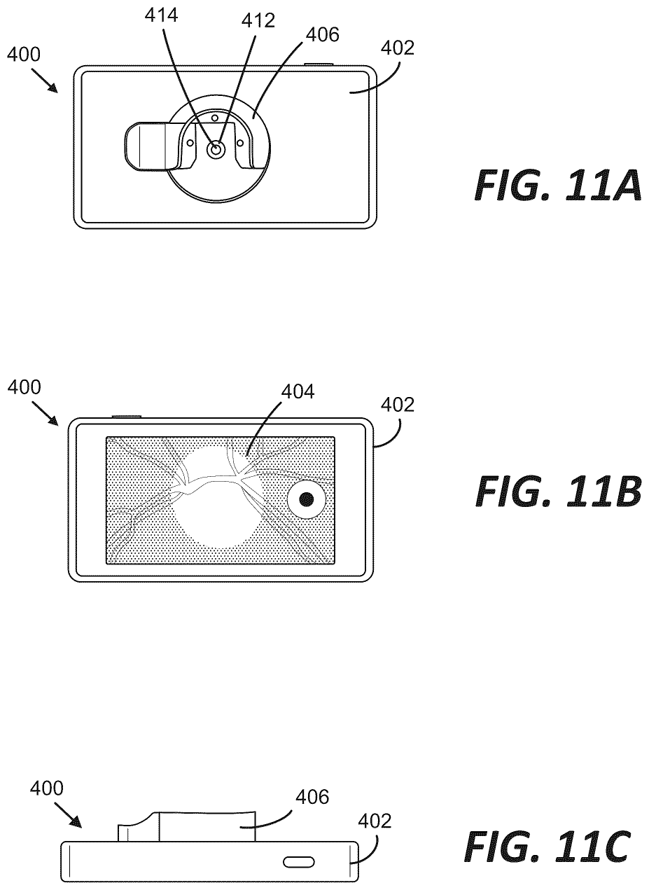

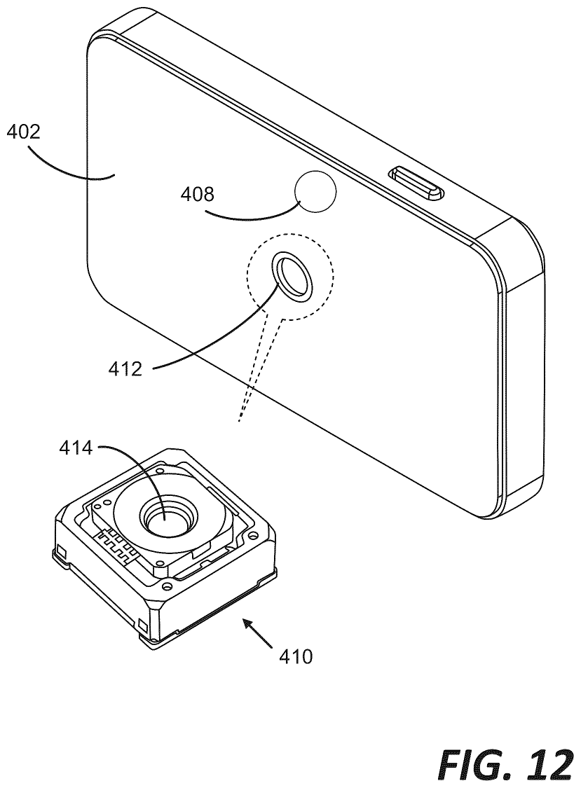

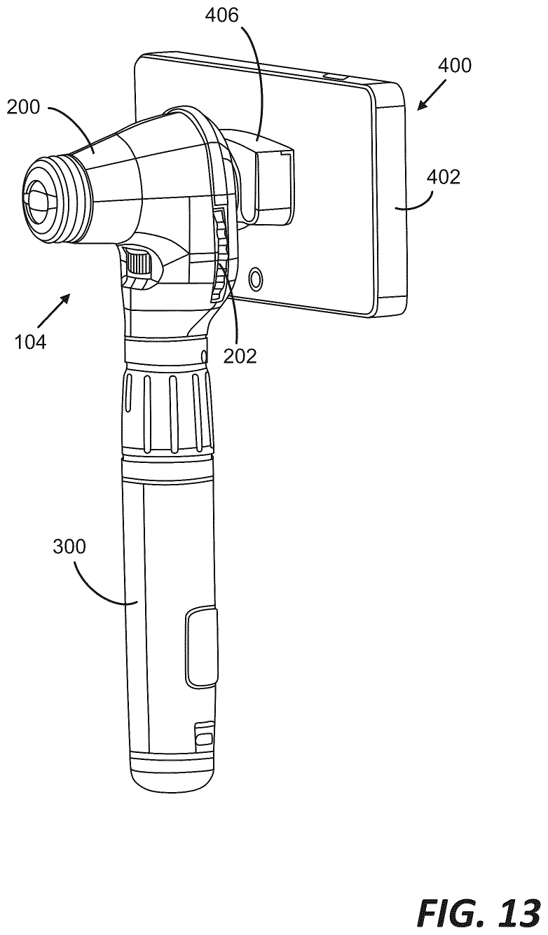

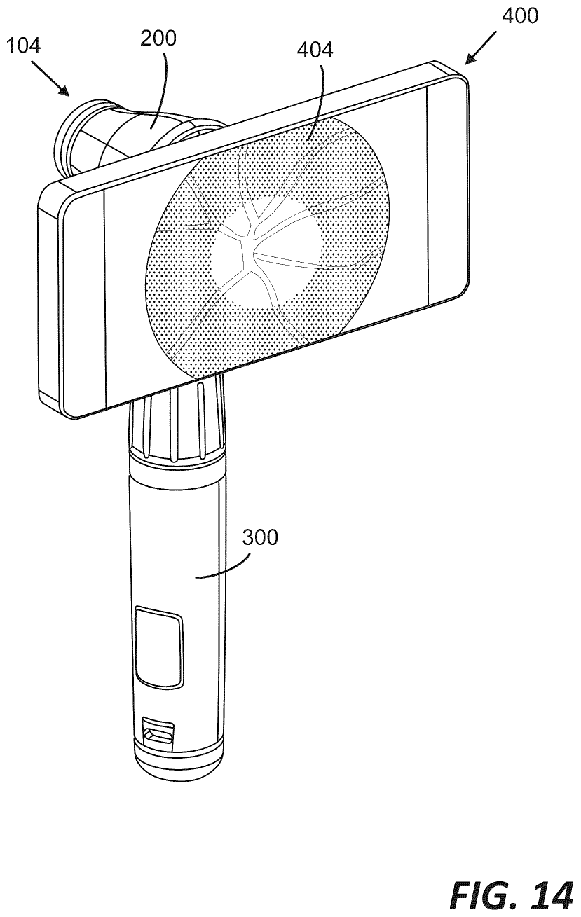

FIG. 9 is an isometric view of an example of the imaging device 400 attached to the ophthalmoscope 104 from a clinician perspective. FIG. 10 is a front isometric view of the imaging device 400. FIG. 11A is a front view of the imaging device 400. FIG. 11B is a rear view of the imaging device 400. FIG. 11C is a top view of the imaging device 400. FIG. 12 is an isometric view showing a camera 410 of the imaging device 400. FIGS. 13 and 14 are respective front isometric and rear isometric views of the imaging device 400 attached to the ophthalmoscope 104. Referring now to FIGS. 9-14, the imaging device 400 captures images viewed from the eyepiece 201 of the ophthalmoscope 104. While FIGS. 9-14 show the imaging device 400 attached to the ophthalmoscope 104, the imaging device 400 can be similarly attached to the otoscope 102, the dermatoscope 106, and additional types of optical viewing devices for capturing images viewed from the eyepieces 201 of those devices.

As shown in FIGS. 9-14, the imaging device 400 includes a housing 402. In this example, a bracket 406 is integrated with a back surface of the housing 402. The bracket 406 allows the imaging device 400 to physically attach to the optical viewing devices 100. For example, the bracket 406 can be fixed around an eyepiece housing 218 (see FIGS. 7 and 8) for attaching the imaging device 400 to an instrument head 200. In alternative examples, the bracket 406 can be part of an accessory case that attaches to the housing 402, and that can be used to physically attach the imaging device 400 to the optical viewing devices 100.

As shown in FIG. 12, the housing 402 further includes an aperture 412 for a lens 414 of the camera 410. The camera 410 is mounted inside the housing 402 of the imaging device 400. When the imaging device 400 is mounted to the instrument head 200, the camera 410 is aligned with the eyepiece 201 of the instrument head 200 for capturing images viewed through the eyepiece 201 of the instrument head 200. The camera 410 is centrally mounted inside the housing 402 to provide even balance and weight distribution for when the imaging device 400 is attached to the instrument head 200, thereby improve the ergonomics of the assembly. A protrusion of the lens 414 beyond the back surface of the housing 402 is minimized such that the lens 414 is substantially flush with the back surface of the housing 402.

The camera 410 can include features such as auto focus, auto-exposure, auto white-balance, and image stabilization. The camera 410 can include a 12MP color image sensor. As an illustrative example, the camera 410 can include an equivalent focal length (on 35 mm film) of 52-77 mm, 4K (30FPS) video recording with 4000×3000 pixel resolution, and a record time of 90 minutes at 4K resolution, 1 minute per clip. Alternative camera parameters are possible.

The housing 402 is compact and lightweight. In some examples, the housing 402 includes a protective overmold having a base layer of plastic material, and a top layer of rubber to provide shock absorption and improved grip. As an illustrative example, the housing 402 can have a thickness (e.g., distance between the lens 414 of the camera 410 and the display screen 404) that is less than 25 mm, and a weight that is less than 250 g.

The housing 402 can include one or more ports such as a USB-C port for charging the battery, and for data transferring including uploading images and videos captured by the camera 410 to another device, such as the wall mounted unit 12. The housing 402 can include a power button to turn on/off and wake up the imaging device 400. The housing 402 houses an integrated, rechargeable battery that can, for example, power 90 minutes of 4K video recording by the camera 410, and 3-4 hours of screen time on the display screen 404.

As shown in FIG. 12, the imaging device 400 can include a wireless antenna 408 that transmits data to another device such as the secondary display screen 14. The wireless antenna 408 can also receive data from another device such as the secondary display screen 14 such as to pair the imaging device 400 to the secondary display screen 14. In some examples, the wireless antenna 408 on the imaging device 400 can transmit and detect radio frequency identification (RFID) signals, near field communication (NFC) signals, Bluetooth signals, Wi-Fi signals, and other types of signals for wireless communications.

The wireless antenna 408 can include an active antenna or tag that activates a passive antenna or tag on the secondary display screen 14 such as to turn on the secondary display screen 14 or to switch the secondary display screen 14 from the patient mode to a clinician mode. When in the clinician mode, the secondary display screen 14 can display the images 405 captured by the camera 410 of the imaging device 400. In some examples, the active antenna activates the passive antenna when in close proximity to the passive antenna such as when the imaging device 400 is positioned in close proximity to the secondary display screen 14.

As shown in FIGS. 9-14, the imaging device 400 includes the display screen 404 for displaying the images 405 captured by the camera 410. In some examples, the display screen 404 is a touchscreen such that it can both display the images, and receive inputs from a user. For example, the display screen 404 can be used by a user of the imaging device to: adjust the settings of the camera 410 (e.g., focus, exposure, white balance, FOV/zoom); tapping the display screen 404 to trigger focus and lock; adjust settings of the display screen 404 such as the screen brightness; provide a virtual keyboard to type in information; display a battery-life indicator; provide video recording controls (e.g., start, stop, save, delete, review, upload; provide a sliding bar to go through video frames, pinch-zoom to enlarge; display arrow(s) to indicate image orientation; and display one or more stamps (e.g., date, time, filter info, etc.) on saved images.

The display screen 404 can include a true color multi-touch screen (in-plane switching (IPS), or light-emitting diode (LED)). The display screen 404 can have a bezel-less design (e.g., full-screen display). The display screen 404 can have a resolution of at least 250 pixels per inch (PPI), a diagonal screen size of about 2 inches to about 5 inches, an aspect ratio of 16:9/4:3, a maximum brightness of 500 nits. The display screen 404 can also include features such as screen auto off, and wake up by power button or tapping the display screen 404.

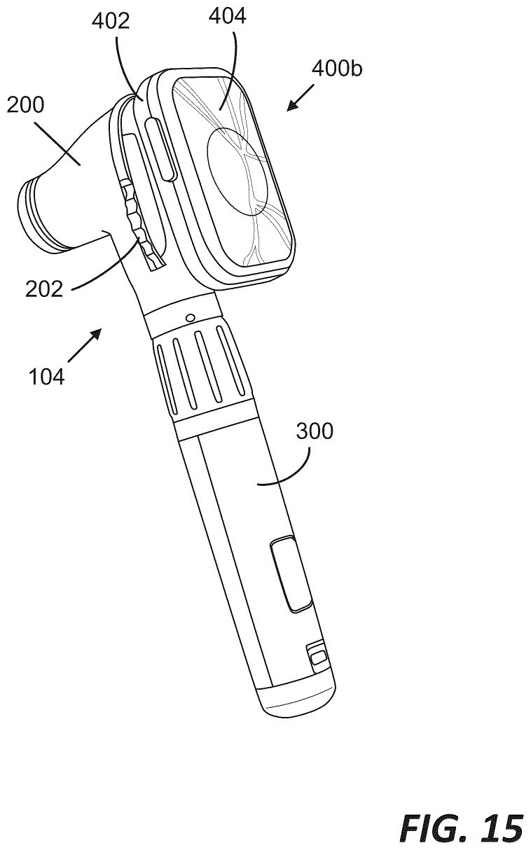

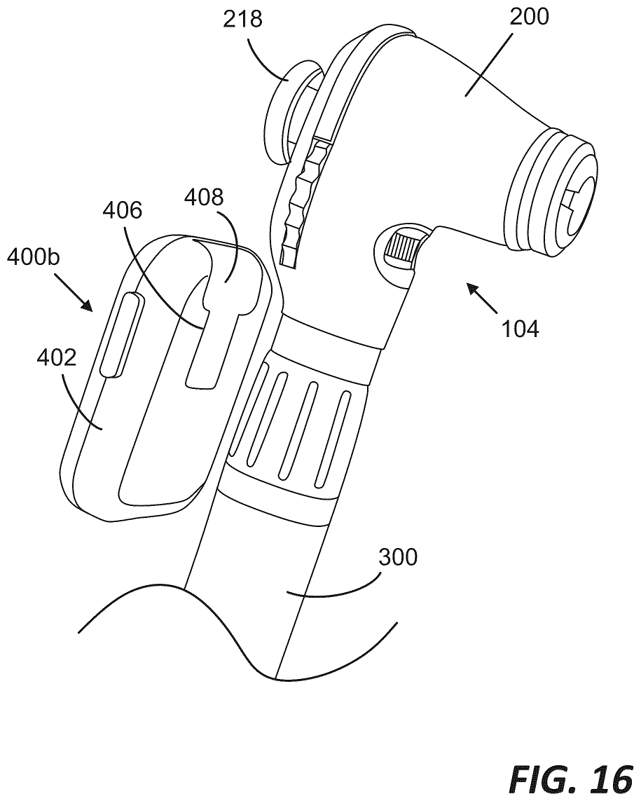

FIG. 15 is an isometric view of another example of an imaging device 400b attached to the ophthalmoscope 104. In FIG. 15, the imaging device 400b is shown from the physician perspective. FIG. 16 is an isometric view of the imaging device 400b before attachment to the ophthalmoscope 104. In FIG. 16, the imaging device 400b is shown from the patient perspective. While FIGS. 15 and 16 show the imaging device 400b attached to the ophthalmoscope 104, the imaging device 400b can similarly attach to the otoscope 102, the dermatoscope 106, and additional types of optical viewing devices.

The imaging device 400b is similar to the imaging device 400 shown in FIGS. 9-14. For example, the imaging device 400b includes a housing 402 having a bracket 406 for physically attaching to the eyepiece housing 218 of the optical viewing device 100. The imaging device 400b similarly includes a display screen 404 for displaying images captured by a camera that is centrally mounted inside the housing 402 of the imaging device 400b to provide even balance and weight distribution. Like in the examples described in FIGS. 9-14, the camera of the imaging device 400b is configured for alignment with the eyepiece 201 of the instrument head 200 for capturing images viewed through the eyepiece 201 of the instrument head 200. The imaging device 400b can also include a wireless antenna 408 for transmitting and detecting data to and from another device such as the secondary display screen 14.

FIG. 17 illustrates an exemplary architecture of a computing device 1700 of the imaging device 400, 400b. The computing device 1700 is used to execute the functionality of the imaging device 400, 400b described herein. The imaging device 400, 400b can include all or some of the elements described with reference to FIG. 17, with or without additional elements.

The computing device 1700 includes at least one processing device 1702. Examples of the at least one processing device 1702 can include central processing units (CPUs), digital signal processors, field-programmable gate arrays, and other types of electronic computing circuits. The at least one processing device 1702 can be part of a processing circuitry having a memory for storing instructions which, when executed by the processing circuitry, cause the processing circuitry to perform the functionalities described herein.

The computing device 1700 also includes a system memory 1704, and a system bus 1706 that couples various system components including the system memory 1704 to the at least one processing device 1702. The system bus 1706 can include any type of bus structure including a memory bus, or memory controller, a peripheral bus, and a local bus.

The system memory 1704 may include a read only memory (ROM) 1708 and a random-access memory (RAM) 1710. An input/output system containing routines to transfer information within the computing device 1700, such as during start up, can be stored in the read only memory (ROM) 1708. The system memory 1704 can be housed inside the housing 402.

The computing device 1700 can further include a secondary storage device 1714 for storing digital data. The secondary storage device 1714 is connected to the system bus 1706 by a secondary storage interface 1716. The secondary storage devices and their computer-readable media provide nonvolatile storage of computer-readable instructions (including application programs and program devices), data structures, and other data for the computing device 1700.

A number of program devices can be stored in secondary storage device 1714 or the system memory 1704, including an operating system 1718, one or more application programs 1720, other program devices 1722, and program data 1724. The system memory 1704 and the secondary storage device 1714 are examples of computer-readable data storage devices.

The computing device 1700 can include one or more input devices such as the display screen 404 (in examples where the display screen 404 is a touch sensitive touchscreen), one or more physical push buttons on the housing 402 of the imaging device 400, and the camera 410. Additional examples of input devices include a microphone 1726, and an accelerometer 1728 for image orientation on the display screen 404. The computing device 1700 can also include output devices such as the display screen 404, and a speaker 1730.

The input and output devices are connected to the at least one processing device 1702 through an input/output interface 1738 coupled to the system bus 1706. The input and output devices can be connected by any number of input/output interfaces, such as a parallel port, serial port, game port, or a universal serial bus. Wireless communication between the input and output devices and the input/output interface 1738 is possible as well, and can include Wi-Fi, Bluetooth, infrared, 802.11a/b/g/n, cellular, or other wireless communications.

In some examples, the display screen 404 is touch sensitive and is connected to the system bus 1706 via an interface, such as a video adapter 1742. The display screen 404 includes touch sensors for receiving input from a user when the user touches the display. Such sensors can be capacitive sensors, pressure sensors, or other touch sensors. The sensors detect contact with the display, and also the location and movement of the contact over time. For example, a user can move a finger or stylus across the display screen 404 to provide inputs.

The computing device 1700 further includes a communication device 1746 configured to establish communication across a network 1752. In some examples, when used in a local area networking environment or a wide area networking environment (such as the Internet), the computing device 1700 is typically connected to the network 1752 through a network interface, such as a wireless network interface 1750. The wireless network interface 1750 can provide Wi-Fi functionality such as for image and video transferring, live streaming, and providing a mobile hotspot. In some further examples, the wireless network interface 1750 can provide Bluetooth connectivity. Other possible examples using other wired and/or wireless communications are possible. For example, the computing device 1700 can include an Ethernet network interface, or a modem for communicating across the network.

In further examples, the communication device 1746 provides short-range wireless communication. The short-range wireless communication can include one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, a Wi-Fi technology, or similar wireless technologies.

The computing device 1700 typically includes at least some form of computer-readable media. Computer-readable media includes any available media that can be accessed by the computing device 1700. By way of example, computer-readable media can include computer-readable storage media and computer-readable communication media.

Computer-readable storage media includes volatile and nonvolatile, removable, and non-removable media implemented in any device configured to store information such as computer-readable instructions, data structures, program devices, or other data. Computer-readable storage media includes, but is not limited to, random access memory, read only memory, electrically erasable programmable read only memory, flash memory or other memory technology, or any other medium that can be used to store the desired information and that can be accessed by the computing device 1700.

Computer-readable communication media embodies computer-readable instructions, data structures, program devices or other data in a modulated data signal such as a carrier wave or other transport mechanism and includes any information delivery media. Modulated data signal refers to a signal having one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are also included within the scope of computer-readable media.

The computing device 1700 is an example of programmable electronics, which may include one or more computing devices, and when multiple computing devices are included, such computing devices can be coupled together with a suitable data communication network so as to collectively perform the various functions, methods, or operations disclosed herein.

The computing device 1700 can include a location identification device 1748. The location identification device 1748 is configured to identify the location or geolocation of the computing device 1700. The location identification device 1748 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

The various embodiments described above are provided by way of illustration only and should not be construed to be limiting in any way. Various modifications can be made to the embodiments described above without departing from the true spirit and scope of the disclosure.

What is claimed is:

1. An optical viewing system, comprising:
an optical viewing device having an eyepiece;
an imaging device including:
a housing having a bracket that is configured to removably attach the imaging device to the optical viewing device;
a camera configured to capture images viewed through the eyepiece; and
a display screen configured to display the images captured by the camera; and
a secondary display screen physically separate from the optical viewing device and the imaging device, the secondary display screen pairing with the imaging device to display the images viewed through the eyepiece of the optical viewing device, and the pairing between the imaging device and the secondary display screen expires when a condition is satisfied.

2. The system of claim 1, wherein the condition is satisfied when the optical viewing device is returned to a storage area or after a predetermined period of time elapses.

3. The system of claim 1, wherein the secondary display screen is included on a wall mounted unit that includes a storage area for the optical viewing device, and one or more physiological parameter measurement devices.

4. The system of claim 3, wherein the one or more physiological parameter measurement devices include at least one of a blood pressure monitor, a pulse oximeter, and a thermometer.

5. The system of claim 1, wherein the secondary display screen receives the images from the imaging device through a wireless connection.

6. The system of claim 1, wherein the pairing between the imaging device and the secondary display screen is established by the camera of the imaging devices scanning a machine readable label displayed on the secondary display screen.

7. The system of claim 6, wherein the machine readable label is dynamically updated at predetermined intervals of time.

8. The system of claim 1, wherein the camera of the imaging device is configured to scan a second machine readable label associated with a patient for displaying an identity of the patient on the secondary display screen.

9. The system of claim 1, wherein the display screen of the imaging device is further configured to display an identity of a patient.

10. The system of claim 1, wherein the camera of the imaging device is configured to scan a machine readable label associated with a clinician, and the imaging device is blocked from streaming the images unless the clinician is authorized to use the imaging device.

11. The system of claim 1, wherein the display screen of the imaging device displays a first code, the secondary display screen displays a second code, and the pairing between the imaging device and the secondary display screen is confirmed when the second code matches the first code.

12. The system of claim 1, wherein the secondary display screen displays content unrelated to the optical viewing device when not paired with the imaging device.

13. The system of claim 1, wherein the imaging device is further configured to transmit the images to a display device remotely located from the optical viewing device and the secondary display screen.

14. The system of claim 1, wherein the imaging device is configured to store the images in an electronic medical record.

15. The system of claim 1, wherein the optical viewing device includes an otoscope, an ophthalmoscope, or a dermatoscope.

16. An imaging device for use with an optical viewing device, the imaging device comprising:
a housing having a bracket for removably attaching the imaging device to the optical viewing device;
a camera for capturing images viewed from an eyepiece of the optical viewing device;
a display screen for displaying the images captured by the camera; and
a communications device for streaming the images viewed from the eyepiece of the optical viewing device to a secondary display screen separately located from the optical viewing device and the imaging device, wherein a pairing between the imaging device and the secondary display screen expires when a condition is satisfied.

17. The imaging device of claim 16, wherein the condition is satisfied when the optical viewing device is returned to a storage area or after a predetermined period of time elapses.

18. The imaging device of claim 16, wherein the communications device transmits the images to the secondary display screen through a wireless connection.

19. The imaging device of claim 16, wherein the bracket allows the imaging device to attach to an otoscope, an ophthalmoscope, or a dermatoscope.

20. An optical viewing system, comprising:
an optical viewing device having an eyepiece; and
an imaging device including:
a housing having a bracket for removably attaching the imaging device to the optical viewing device;
a camera for capturing images viewed through the eyepiece;
a display screen for displaying the images captured by the camera; and
a communications device for streaming the images viewed from the eyepiece of the optical viewing device to a secondary display screen separately located from the optical viewing device and the imaging device, wherein a pairing between the imaging device and the secondary display screen expires when a condition is satisfied.
